(19) United States
(12) Patent Application Publication
(10) Pub. No.: US 2022/0235073 A1
(43) Pub. Date: Jul. 28, 2022

(54) ARTEMISININ-DERIVATIVE N-HETEROCYCLIC CARBENE GOLD(I) HYBRID COMPLEXES

(71) Applicants: CENTRE NATIONAL DE LA RECHERCHE SCIENTIFIQUE, Paris (FR); UNIVERSITE PAUL SABATIER TOULOUSE III, Toulouse Cedex 9 (FR)

(72) Inventors: Catherine HEMMERT, Pompertuzat (FR); Heinz GORNITZKA, Pompertuzat (FR); Olivier CUVILLIER, Vielle Toulouse (FR); Marie-Lise MADDELEIN, Vielle Toulouse (US)

(21) Appl. No.: 17/622,042

(22) PCT Filed: Jun. 27, 2019
(86) PCT No.: PCT/IB2019/000823

§ 371 Date: Dec. 22, 2021

(51) Int. Cl. C07F 1/00; A61K 45/06; A61K 31/28; A61P 35/00

(52) U.S. Cl. CPC C07F 1/00; A61P 35/00; A61K 31/28; A61K 45/06

(57) ABSTRACT

The present invention relates to compounds of formula (I), which are artemisinin-derivative N-heterocyclic carbene gold (1) hybrid complexes, and to their therapeutic uses.

The present invention concerns novel specific artemisinin-derivative N-heterocyclic carbene gold(I) hybrid complexes and their uses in therapy.

Resistance to chemotherapy and radiotherapy remains a major obstacle in the successful treatment of cancer. Resistance may occur during cancer treatment because of many reasons, such as some of the cancer cells which are not killed can mutate and become resistant, gene amplification resulting in the overexpression of a protein that renders the treatment ineffective may occur, or cancer cells may develop a mechanism to inactivate the treatment.

Nuclear factor erythroid-2 related factor 2 (Nrf2 or NFE2L2) is a redox-sensitive transcription factor that regulates the expression of electrophile and xenobiotic detoxification enzymes and efflux proteins, which confer cytoprotection against oxidative stress and apoptosis in normal cells.

Cancer cells show greater expression of drug detoxification enzymes and efflux pumps. This characteristic can result in cancer therapeutic resistance due to the ability of a cancer cell to eliminate a toxic drug—such as a chemotherapeutic drug—from the cell. Further, a gain of Nrf2 in cancer can cause an increased expression of drug detoxification enzymes and efflux pumps. Nrf2 is overexpressed in tumors which are resistant to chemo- and radiotherapy.

Thus, Nrf2 is a target of choice in the treatment of cancer and in restoring sensibility to conventional treatments.

Among the different anticancer drugs currently on the market or under experiment, some of them are already known and/or used for a different therapeutic purpose, and are under repositioning.

Currently, artemisinin (ART) and its derivatives represent the most important class of drugs to combat malaria. In the period from 2010 to 2017, the number of deaths worldwide caused by malaria decreased by 28% mainly due to the use of ART-based combination therapies (ACTs).

However, the interest on ART derivatives is not only limited to malaria, as it has been shown that this kind of molecules shows interesting activities against viral diseases and cancer.

In the case of cancer, one mechanism of action is based on reactive oxygen species (ROS) formation, due to an activation of ART derivatives by iron from free heme or via ferroptosis. This activation takes mainly place in mitochondria, where fresh heme is produced continuously. It has been evidenced that mitochondria-targeting ART derivatives show stronger anticancer activities than non-mitochondria-targeting ones.

Nevertheless, the efficacy of ART in cancer is still not optimal.

Besides, cationic N-heterocyclic carbene (NHC) gold(I) complexes show good anticancer activities and the main mechanism of action discussed concerns apoptosis due to an antimitochondrial activity of such complexes. Among the different gold complexes which are described, auranofin is the prototype of said family. Auranofin is authorized for the treatment of rhumatoid arthritis, but its repositioning in oncology, as well as in other parthologies, is currently under investigation.

Therefore, there is a need for the development of novel and efficient anti-cancer drugs, which would be efficient while being specific for tumoral tissues. Especially, there is a need for novel and efficient anti-cancer drugs useful for restoring sensibility to conventional treatments and/or for decreasing resistance of cancer cells to chemo- or radiotherapy.

The present invention proposes new artemisinin-gold complexes which aim to solve these needs:

Indeed, as shown in the example, the inventors have discovered that cationic bisNHC gold(I) complexes incorporating an ether derivative of dihydroartemisinin are cytotoxic and selective for cancerous tissues. These complexes are hybrid because they comprise both the cationic NHC gold(I) complex and an ether derivative of dihydroartemisinin, which is fused to the complex via a linker (“hybrid complexes”).

Said hybrid complexes show an antitumoral activity with IC₅₀ in the order of nM, are specific towards tumoral cells, and show higher anti-tumoral activity than artemisinin alone and than auranofin alone.

Moreover, without being bound by any theory, the mechanism of action of said hybrid complexes seems original: they inhibit the transcriptional activity of Nrf2, which is the key transcription factor involved in detoxification and elimination of ROS.

As shown in the example, surprisingly, the hybrid complexes inhibit the activity of Nrf2 in any dose, whereas each one of artemisinin, auranofin or the cationic bisNHC gold(I) complex alone (i.e. without artemisinin, as exemplified by complex 3) all activate Nrf2.

Consequently, the present invention first relates to a compound chosen from compounds of formula (I) and their isomers:

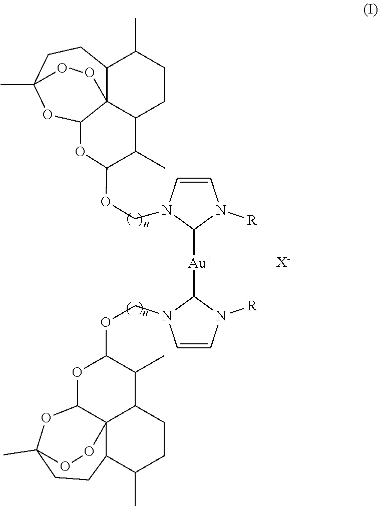

wherein:
each R is independently a C1-C6 alkyl, quinoline, benzyl or mesityl,
X⁻ is an anion, and
n is a integer which is equal to 3, 4 or 5.

By isomers, it is meant alpha and beta isomers. By alpha and beta isomers, it is meant that the compounds of formula (I) have dihydroartemisinin in alpha or beta conformation, respectively.

Preferably, the compounds of formula (I) are beta isomers, which are compounds of formula (I′). Thus, preferably the present invention relates to a compound chosen from compounds of formula (I′):

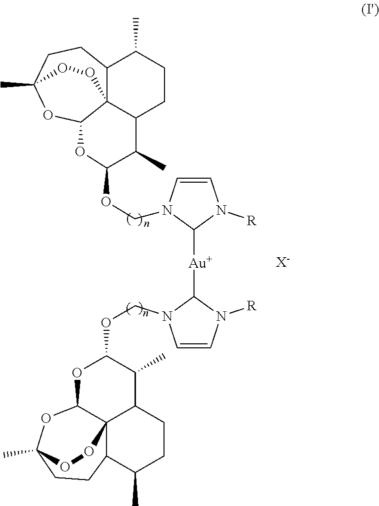

wherein:
each R is independently a C1-C6 alkyl, quinoline, benzyl or mesityl,
X⁻ is an anion, and
n is a integer which is equal to 3, 4 or 5.

The compounds of formula (I) according to the invention correspond to cationic bisNHC gold(I) complexes with an ether derivative of dihydroartemisinin (DHA). DHA is a semi-synthetic derivative of ART and a metabolite of all ART compounds.

ART and DHA respectively correspond to compounds of formula (II) and (III) below:

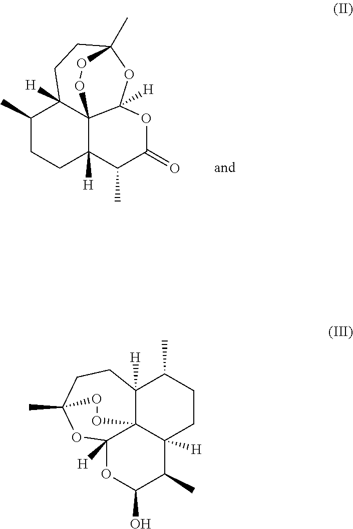 [SMILES: C[C@@H]1CC[C@H]2[C@@H](C)C(=O)O[C@@H]3O[C@@]4(C)CC[C@@H]1[C@]32OO4 ; C[C@H]1[C@@H](O)O[C@@H]2O[C@]3(C)CC[C@H]4[C@H](C)CC[C@@H]1[C@@]24OO3]

The compounds of formula (I) according to the invention comprise two R radicals, which may be identical or different, and which are chosen from methyl, isopropyl, quinoline, benzyl and mesityl radicals.

By “C1-C6 alkyl”, it is meant a linear hydrocarbon group comprising from 1 to 6 carbon atoms, or a branched hydrocarbon group comprising from 3 to 6 carbon atoms. Examples of C1-C6 alkyl groups include methyl, ethyl, n-propyl, isopropyl, n-butyl, isobutyl, tert-butyl, n-pentyl, isopentyl and n-hexyl groups, and preferably methyl, n-butyl, n-pentyl, n-hexyl, isopropyl or tert-butyl. More preferably, the C1-C6 alkyl is methyl or isopropyl.

By quinoline radical, it is meant a radical:

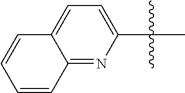 [SMILES: *c1ccc2ccccc2n1]

By benzyl radical, it is meant the radical CH2-phenyl, wherein the phenyl is not substituted.

Finally, by mesityl radical, it is meant the radical of formula (a) below:

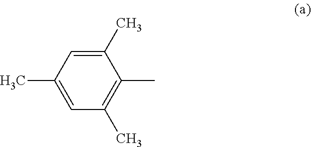

Preferably both R radicals are identical.

Preferably both R are methyl. Alternatively, preferably both R are isopropyl.

Preferably X⁻ is an anion chosen from halogens, nitrate and hexafluorophosphate, preferably from chloride (Cl⁻) and nitrate (NO₃⁻).

Preferably, the compound of formula (I) of the invention is chosen from the following compounds:

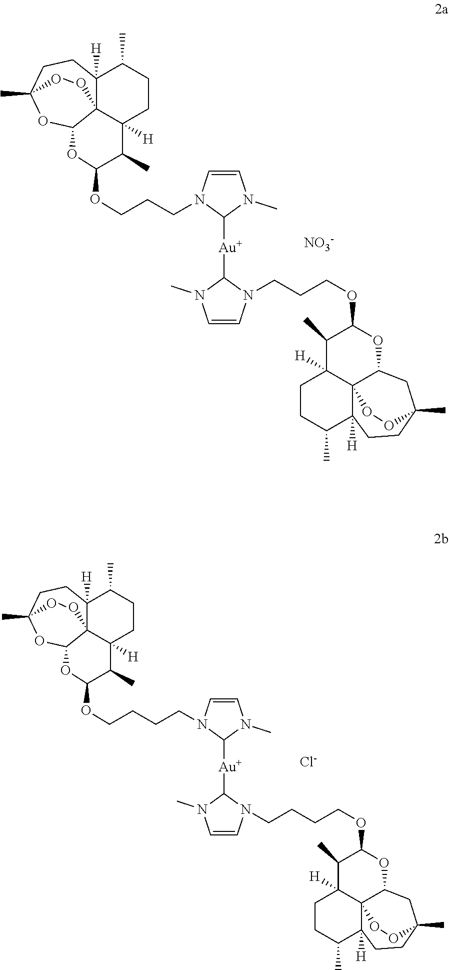

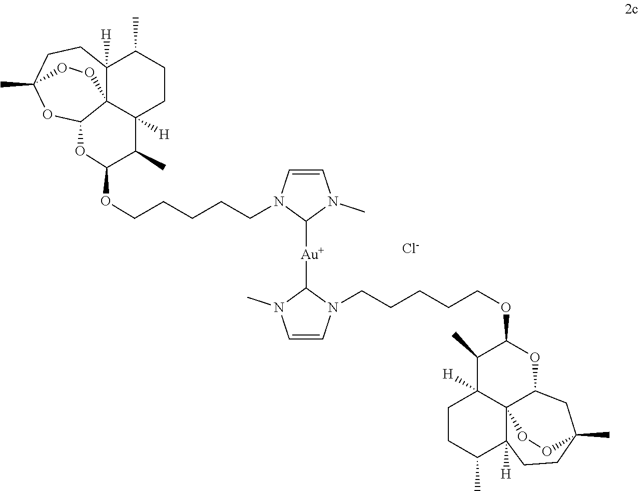

Compound 2a is the compound of formula (I) wherein both R are methyl, X⁻ is NO₃⁻ and n=3.

Compound 2b is the compound of formula (I) wherein both R are methyl, X⁻ is Cl⁻ and n=4.

Compound 2c is the compound of formula (I) wherein both R are methyl, X⁻ is Cl⁻ and n=5.

As shown in the examples, the compounds of formula (I) of the invention are cytotoxic and are selective for cancerous tissues. Indeed, as shown in Tables 1 to 3, said compounds are very specific for cancerous cell lines of various cancers (i.e. prostate, breast, liver, bone, bladder, lung and leukemia) as compared to non-cancerous cell lines (epithelial cells from prostate, fibroblasts and osteoblasts).

Moreover, as shown in Table 3, the compounds of formula (I) of the invention surprisingly show an antitumoral activity which is significantly higher than the one of artemisinin alone, than the one of bisNHC-gold(I) complex 3 alone (without artemisinin or DHA), than the one of auranofin alone and than the one of a mixture (in a respective molar ratio of 1:2) of bisNHC-gold(I) complex 3 alone and DHA alone.

Interestingly, the compounds of the invention also show interesting anti-inflammatory properties. Indeed, they show an inhibitory effect of the NF-κB pathway, which is the central pathway of inflammatory responses regulating the innate and adaptive immune functions.

They specifically show an inhibitory effect of the NF-κB transcription factor induced by TNFalpha in a dose-dependent manner: for example, compound 2a shows an IC₅₀ of around 615 nM, which is much lower than the IC₅₀ for auranofin which is 2.96 μM, and than the IC₅₀ for dihydroartemisinin which is 8.91 μM.

### Preparation of the Compounds of the Invention

The compounds of the invention may be prepared by the following process, which is illustrated in Scheme 1 of the example:
a first step of reacting DHA with a bromoalcohol, preferably in the presence of a catalyst, in order to obtain an ether which corresponds to the single β-isomer DHA-C3 to DHA-C5;
a second step of reacting the compound obtained in the first step with methyl imidazole in order to obtain the corresponding carbene precursors (i.e. such as proligands 1a to 1c); and
a third step of obtaining the compounds of formula (I):
   For the compounds of formula (I) with n=3, a transmetalation route using Ag₂O, followed by an ion exchange with AgNO₃ and subsequent addition of Au(SMe₂)Cl is applied;
   For the compounds of formula (I) with n=4 or 5, the direct metalation using K₂CO₃ and Au(SMe₂)Cl is applied.

In details, DHA and NHCs precursors are fused by using aliphatic linkers of different lengths C3 to C5 (according to the definition of n in formula (I)).

The synthesis starts with the formation of an ether, by reacting DHA (which is commercially available) with a bromoalcohol, preferably in the presence of a catalyst such as boron trifluoride etherate catalyst and according to the procedure described by Haynes (see reference 1) for the C3-derivative, leading to the single β-isomer DHA-C3 to DHA-C5.

The next step is the reaction between the bromoalkyl DHA derivatives and methyl imidazole in order to obtain the corresponding carbene precursors, such as proligands 1a to 1c.

The formation of the target gold complexes is achieved by two approaches:
For the compounds of formula (I) with n=3, a transmetalation route involving the mild base Ag₂O, followed by an ion exchange with AgNO₃ and subsequent addition of Au(SMe₂)Cl is used;
For the compounds of formula (I) with n=4 or 5, the direct metalation involving K₂CO₃ and Au(SMe₂)Cl is applied.

### Proligands of the Compounds of the Invention

The present invention also relates to proligands of the compounds of formula (I), which are as defined in formula (IV) below.

Thus, the present invention relates to a compound chosen from compounds of formula (IV) and their isomers:

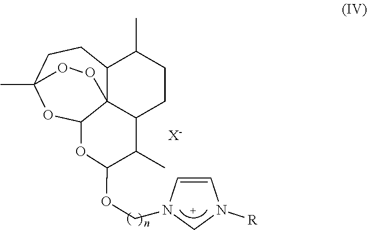

wherein:
each R is independently a C1-C6 alkyl, quinoline, benzyl or mesityl,
X⁻ is an anion, and
n is a integer which is equal to 3, 4 or 5.

By isomers, it is meant alpha and beta isomers. By alpha and beta isomers, it is meant that the compounds of formula (IV) have dihydroartemisinin in alpha or beta conformation, respectively. Preferably, the compounds of formula (IV) are beta isomers, which are compounds of formula (IV′).

Thus, preferably, the present invention relates to a compound of formula (IV′):

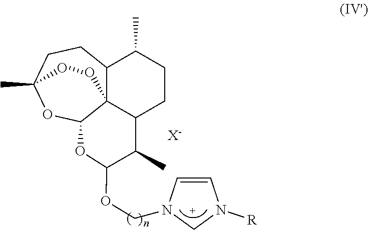

All the above definitions for the compounds of formula (I) are also applicable to the compounds of formula (IV) and (IV′).

Preferably both R radicals are identical, and preferably are methyl or isopropyl.

Preferably X⁻ is an anion chosen from halogens, nitrate and hexafluorophosphate, preferably bromide (Br).

Preferably, the compound of formula (IV) or (IV′) is chosen from 3′-methyl-1′-[10β-(20-propoxy)dihydroartemisinin]1H-imidazol-3-ium halide, 3′-methyl-1′-[10β-(21-butoxy)dihydroartemisinin]1H-imidazol-3-ium halide and 3′-methyl-1′-[10β-(22-pentoxy)dihydroartemisinin]1H-imidazol-3-ium halide.

Preferably, the compound of formula (IV) or (IV′) is chosen from 3′-methyl-1′-[10β-(20-propoxy)dihydroartemisinin]1H-imidazol-3-ium bromide, 3′-methyl-1′-[10β-(21-butoxy)dihydroartemisinin]1H-imidazol-3-ium bromide and 3′-methyl-1′-[10β-(22-pentoxy)dihydroartemisinin]1H-imidazol-3-ium bromide. These compounds are described in the example as proligands 1a, 1b and 1c, respectively.

### Composition and Uses

The present invention also relates to a composition comprising, in a pharmaceutically acceptable medium, at least one compound of formula (I) according to the invention.

The present invention also relates to the use of a compound of formula (I) according to the invention as a medicament.

The present invention also relates to the use of a compound of formula (I) according to the invention for preventing and/or treating cancer.

The present invention also relates to the use of a compound of formula (I) according to the invention for preventing and/or treating inflammation.

### Anticancer Use

The compounds of formula (I) of the invention may be used for preventing and/or treating cancer.

By “preventing”, it is meant avoiding the cancer to occur.

By “treatment”, it is meant the curative treatment of cancer. A curative treatment is defined as a treatment that completely treat (cure) or partially treat cancer (i.e. induces tumor growth stabilization, retardation or regression).

The “subject” refers to any subject and typically designates a patient, preferably a subject undergoing a treatment of cancer such as immunotherapy, chemotherapy and/or radiotherapy. In any case, the subject is preferably a vertebrate, more preferably a mammal, even more preferably a human being.

By “cancer”, it is meant any type of cancer. The cancer may be solid or non solid, and may be for example selected from a colon cancer, a colorectal cancer, a melanoma, a bone cancer, a breast cancer, a thyroid cancer, a prostate cancer, an ovarian cancer, a lung cancer, a pancreatic cancer, a glioma, a cervical cancer, an endometrial cancer, a head and neck cancer, a liver cancer, a bladder cancer, a renal cancer, a skin cancer, a stomach cancer, a testis cancer, an urothelial cancer or an adrenocortical carcinoma, leukemia but also non solid cancers such as lymphoma.

Preferably, the cancer is a breast cancer, a prostate cancer, a lung cancer, a liver cancer, a bone cancer, a bladder cancer or a leukemia.

The cancer can be a metastatic cancer or not. A typical cancer is a cancer resistant to the first-line chemotherapy.

The invention also relates to the use of at least one compound of formula (I) for increasing the sensitivity of a cancer to a chemotherapeutic drug.

A further object of the invention is the use of at least one compound of formula (I) for decreasing the resistance of a cancer with respect to a chemotherapeutic drug.

The invention also relates to a product comprising:

a) at least one compound of formula (I) of the invention, and
b) at least one additional therapy,
as a combination product for a simultaneous, separate or sequential use for treating cancer, and/or for preventing cancer metastasis, and/or for preventing cancer recurrence, and/or for decreasing resistance to the additional therapy b), in a subject.

It also relates to the use of at least one compound of formula (I) of the invention, for preventing and/or treating a cancer in combination or in association with at least one additional therapy.

It further relates to the use of at least one compound of formula (I) of the invention, for preventing and/or treating a cancer in a subject treated by at least one additional therapy. The invention also relates to at least one compound of formula (I) of the invention, for use as an adjuvant cancer therapy. An adjuvant therapy is a therapy for treating cancer that is given besides a primary or initial therapy (“first-line therapy”), to maximize its effectiveness.

Said additional therapy b) may be immunotherapy, chemotherapy and/or radiotherapy. Preferably the additional therapy b) is immunotherapy and/or chemotherapy.

By “immunotherapy”, it is meant a therapy with is able to induce, enhance or suppress an immune response. Said immunotherapy is preferably chosen from cytokines, chemokines, growth factors, growth inhibitory factors, hormones, soluble receptors, decoy receptors; monoclonal or polyclonal antibodies, mono-specific, bi-specific or multi-specific antibodies, monobodies, polybodies; vaccination; or adoptive specific immunotherapy.

Preferably the immunotherapy is chosen from monoclonal or polyclonal antibodies, mono-specific, bi-specific or multi-specific antibodies, monobodies, polybodies, such as anti-angiogenic agents like Bevacuzimab (mAb, inhibiting VEGF-A, Genentech); IMC-1121B (mAb, inhibiting VEGFR-2, ImClone Systems); CDP-791 (Pegylated DiFab, VEGFR-2, Celltech); 2C3 (mAb, VEGF-A, Peregrine Pharmaceuticals); VEGF-trap (soluble hybrid receptor VEGF-A, PIGF (placenta growth factor) Aventis/Regeneron).

Preferably the immunotherapy is a monoclonal antibody, preferably an anti-checkpoint antibody.

The anti-checkpoint antibodies comprise antibodies directed against an immune checkpoint, which may be chosen from PD1, PDL1, PDL2, CTLA4, BTLA, CD27, CD40, OX40, GITR (also called “Tumor necrosis factor receptor superfamily member 18” or TNFRSF18), CD137 (also called 4-1BB or TNFRS9), CD28, ICOS, IDO (indoleamine 2,3-dioxygenase), B7H3 (also called CD276), KIR2DL2 (also called killer cell immunoglobulin-like receptor 2DL2), NKG2 (a family of the C-type lectin receptors), LAGS (also called Lymphocyte Activation Gene-3) and CD70. Preferably the anti-checkpoint antibodies are anti-PD1, anti-PDL1, anti-PDL2 or anti-CTLA4 antibodies. Anti-PD1 antibodies include nivolumab and pembrolizumab. Anti-CTLA4 antibodies include ipilimumab and tremelimumab.

The “chemotherapy” or “chemotherapeutic agent” refers to compounds which are used in the treatment of cancer and that have the functional property of inhibiting a development or progression of a neoplasm in a human, particularly a malignant (cancerous) lesion.

Chemotherapeutic agents have different modes of actions, for example, by influencing either DNA or RNA and interfering with cell cycle replication.

Examples of chemotherapeutic agents that act at the DNA level or on the RNA level are:
anti-metabolites, such as Azathioprine, Cytarabine, Fludarabine phosphate, Fludarabine, Gemcitabine, cytarabine, Cladribine, capecitabine 6-mercaptopurine, 6-thioguanine, methotrexate, 5-fluoroouracil and hyroxyurea;
alkylating agents, such as Melphalan, Busulfan, Cisplatin, Carboplatin, Cyclophosphamide, Ifosphamide, Dacarabazine, Fotemustine, Procarbazine, Chlorambucil, Thiotepa, Lomustine, Temozolomide;
anti-mitotic agents, such as Vinorelbine, Vincristine, Vinblastine, Docetaxel, Paclitaxel;
topoisomerase inhibitors, such as Doxorubincin, Amsacrine, Irinotecan, Daunorubicin, Epirubicin, Mitomycin, Mitoxantrone, Idarubicin, Teniposide, Etoposide, Topotecan;
antibiotics, such as actinomycin and bleomycin;
asparaginase;
anthracyclines or taxanes.

Other chemotherapeutic agents are tyrosine kinase inhibitors (TKI). A number of TKIs are in late and early stage development for treatment of various types of cancer. Examplary TKIs include, but are not limited to: BAY 43-9006 (Sorafenib, Nexavar®) and SU11248 (Sunitinib, Sutent®), imatinib mesylate (Gleevec®, Novartis); Gefitinib (Iressa®, AstraZeneca); Erlotinib hydrochloride (Tarceva®, Genentech); Vandetanib (Zactima®, AstraZeneca), Tipifarnib (Zarnestra®, Janssen-Cilag); Dasatinib (Sprycel®, Bristol Myers Squibb); Lonafarnib (Sarasar®, Schering Plough); Vatalanib succinate (Novartis, Schering AG); Lapatinib (Tykerb®, GlaxoSmithKline); Nilotinib (Novartis); Lestaurtinib (Cephalon); Pazopanib hydrochloride (GlaxoSmithKline); Axitinib (Pfizer); Canertinib dihydrochloride (Pfizer); Pelitinib (National Cancer Institute, Wyeth); Tandutinib (Millennium); Bosutinib (Wyeth); Semaxanib (Sugen, Taiho); AZD-2171 (AstraZeneca); VX-680 (Merck, Vertex); EXEL-0999 (Exelixis); ARRY-142886 (Array BioPharma, AstraZeneca); PD-0325901 (Pfizer); AMG-706 (Amgen); BIBF-1120 (Boehringer Ingelheim); SU-6668 (Taiho); CP-547632 (OSI); (AEE-788 (Novartis); BMS-582664 (Bristol-Myers Squibb); JNK-401 (Celgene); R-788 (Rigel); AZD-1152 HOPA (AstraZeneca); NM-3 (Genzyme Oncology); CP-868596 (Pfizer); BMS-599626 (Bristol-Myers Squibb); PTC-299 (FTC Therapeutics); ABT-869 (Abbott); EXEL-2880 (Exelixis); AG-024322 (Pfizer); XL-820 (Exelixis); OSI-930 (OSI); XL-184 (Exelixis); KRN-951 (Kirin Brewery); CP-724714 (OSI); E-7080 (Eisai); HKI-272 (Wyeth); CHIR-258 (Chiron); ZK-304709 (Schering AG); EXEL-7647 (Exelixis); BAY-57-9352 (Bayer); BIBW-2992 (Boehringer Ingelheim); AV-412 (AVEO); YN-968D1 (Advenchen Laboratories); Staurosporin, Midostaurin (PKC412, Novartis); Perifosine (AEterna Zentaris; Keryx, National Cancer Institute); AG-024322 (Pfizer); AZD-1152 (AstraZeneca); ON-01910Na (Onconova); and AZD-0530 (AstraZeneca).

Herein described are also (i) a method for preventing or treating cancer, (ii) a method for increasing the sensitivity of a cancer to a chemotherapeutic agent, and (iii) a method for decreasing the resistance of a cancer with respect to a chemotherapeutic drug, each of said methods comprising administering to a subject in need thereof with an effective amount of at least one compound of formula (I) as defined above, preferably together with a chemotherapeutic drug.

### Anti-Inflammatory Use

The compounds of formula (I) of the invention may be used for preventing and/or treating inflammation.

By “preventing”, it is meant avoiding the inflammation to occur.

By “treatment”, it is meant the curative treatment of inflammation. A curative treatment is defined as a treatment that completely treat (cure) or partially treat inflammation.

The “subject” refers to any subject and typically designates a patient afflicted by inflammation, or a subject undergoing a treatment of inflammatory disease, or a subject at risk, or suspected to be at risk, of developing an inflammatory disease. In any case, the subject is preferably a vertebrate, more preferably a mammal, even more preferably a human being.

The inflammatory disease is preferably a chronic inflammatory disease, and may be chosen from rheumatoid arthritis, Crohn's disease, inflammatory bowel disease (IBD), osteoartrosis, osteoporosis, dermatitis, psoriasis, asthma, respiratory distress syndrome and chronic obstructive pulmonary disease (COPD).

Herein described is also a method for preventing and/or treating an inflammatory disease, comprising administering to a subject in need thereof with an effective amount of at least one compound of formula (I) as defined above.

The compound of formula (I) of the invention is preferably administered at a therapeutically effective amount or dose. As used herein, “a therapeutically effective amount or dose” refers to an amount of the compound of the invention which prevents, removes, slows down the disease, or reduces or delays one or several symptoms or disorders caused by or associated with said disease in the subject, preferably a human being. The effective amount, and more generally the dosage regimen, of the compound of the invention and pharmaceutical compositions thereof may be determined and adapted by the one skilled in the art. An effective dose can be determined by the use of conventional techniques and by observing results obtained under analogous circumstances. The therapeutically effective dose of the compound of the invention will vary depending on the disease to be treated or prevented, its gravity, the route of administration, any co-therapy involved, the patient's age, weight, general medical condition, medical history, etc.

Typically, the amount of the compound to be administered to a patient may range from about 0.01 to 500 mg/kg of body weight for a human patient. In a particular embodiment, the pharmaceutical composition according to the invention comprises 0.01 mg/kg to 300 mg/kg of the compound of the invention, preferably from 0.01 mg/kg to 3 mg/kg, for instance from 25 to 300 mg/kg.

In a particular aspect, the compounds of the invention can be administered to the subject by parenteral route, topical route, oral route or intravenous injection. The compound or the nanoparticle of the invention may be administered to the subject daily (for example 1, 2, 3, 4, 5, 6 or 7 times a day) during several consecutive days, for example during 2 to 10 consecutive days, preferably from 3 to 6 consecutive days. Said treatment may be repeated during 1, 2, 3, 4, 5, 6 or 7 weeks, or every two or three weeks or every one, two or three months. Alternatively, several treatment cycles can be performed, optionally with a break period between two treatment cycles, for instance of 1, 2, 3, 4 or 5 weeks. The compound of the invention can for example be administered as a single dose once a week, once every two weeks, or once a month. The treatment may be repeated one or several times per year. Doses are administered at appropriate intervals which can be determined by the skilled person. The amount chosen will depend on multiple factors, including the route of administration, duration of administration, time of administration, the elimination rate of the compound, or of the various products used in combination with said compound, the age, weight and physical condition of the patient and his/her medical history, and any other information known in medicine.

The administration route can be oral, topical or parenteral, typically rectal, sublingual, intranasal, intra-peritoneal (IP), intra-venous (IV), intra-arterial (IA), intra-muscular (IM), intra-cerebellar, intrathecal, intratumoral and/or intradermal. The pharmaceutical composition is adapted for one or several of the above-mentioned routes. The pharmaceutical composition is preferably administered by injection or by intravenous infusion of suitable sterile solutions, or in the form of liquid or solid doses via the alimentary canal.

The present invention also relates to a composition comprising, in a pharmaceutically acceptable medium, at least one compound of formula (I) according to the invention. Such a composition comprises a pharmaceutically acceptable medium (or carrier).

The carrier must be “acceptable” in the sense of being compatible with the other ingredients of the formulations and not deleterious to the recipient thereof.

The pharmaceutical composition can be formulated as solutions in pharmaceutically compatible solvents or as gels, oils, emulsions, suspensions, or dispersions in suitable pharmaceutical solvents or vehicles, or as pills, tablets, capsules, powders, suppositories, etc. that contain solid vehicles in a way known in the art, possibly through dosage forms or devices providing sustained and/or delayed release. For this type of formulation, an agent such as cellulose, lipids, carbonates or starches are used advantageously.

Agents or vehicles that can be used in the formulations (liquid and/or injectable and/or solid) are excipients or inert vehicles, i.e. pharmaceutically inactive and non-toxic vehicles.

Mention may be made, for example, of saline, physiological, isotonic and/or buffered solutions, compatible with pharmaceutical use and known to those skilled in the art. The compositions may contain one or more agents or vehicles chosen from dispersants, solubilizers, stabilizers, preservatives, etc.

Particular examples are methylcellulose, hydroxymethylcellulose, carboxymethylcellulose, cyclodextrins, polysorbate 80, mannitol, gelatin, lactose, liposomes, vegetable oils or animal, acacia, etc. Preferably, vegetable oils are used.

Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

Formulations suitable for parenteral administration conveniently comprise a sterile oily or aqueous preparation of the active ingredient which is preferably isotonic with the blood of the recipient. Every such formulation can also contain other pharmaceutically compatible and non-toxic auxiliary agents, such as, e.g. stabilizers, antioxidants, binders, dyes, emulsifiers or flavoring substances.

The figures used in the present application are the following and serve as illustrative purposes only:

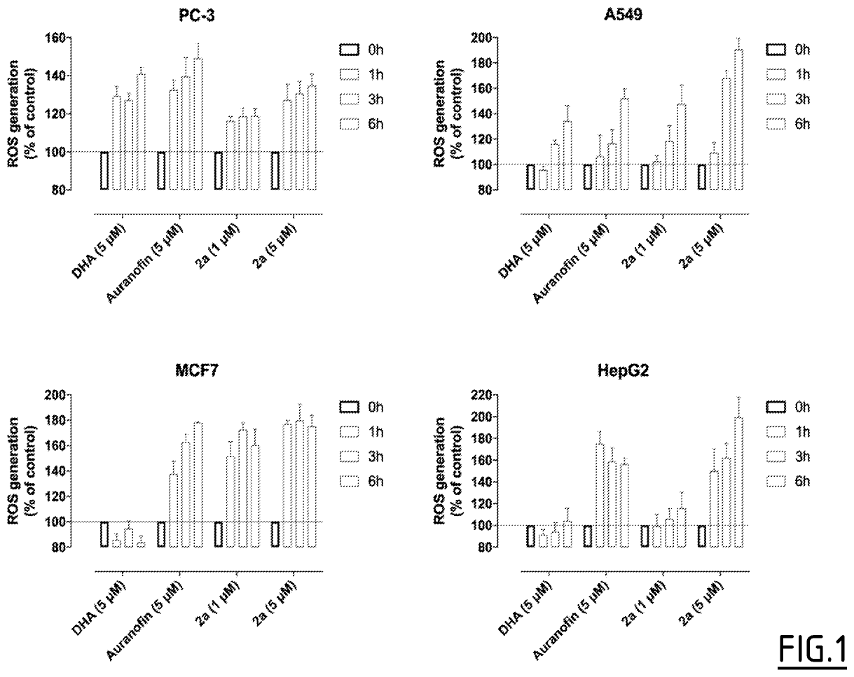

FIG. 1. Induction of ROS by dihydroartemisinin (DHA) and gold complexes (auranofin and complex 2a) on PC-3, A549, MCF-7 and HepG2 cells after different times of treatment. *p<0.05, **p<0.005, ***p<0.001, compared with ROS generation at 0 h.

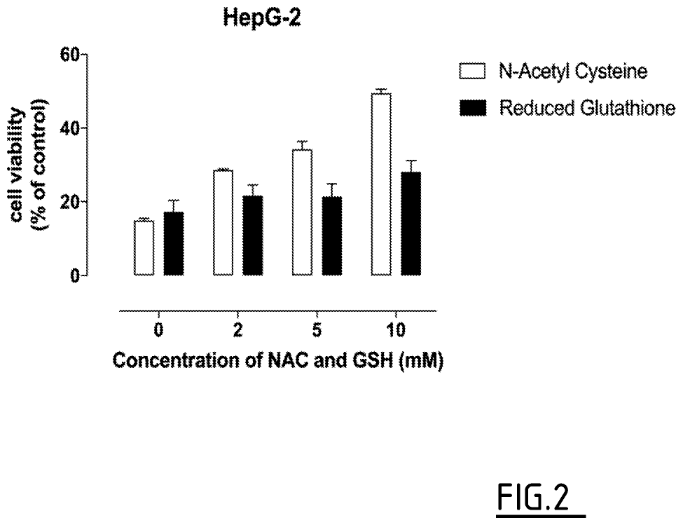

FIG. 2. Impact of N-Acetyl-L-cysteine (NAC) and reduced glutathione (GSH) on the cytotoxicity of complex 2a. HepG2 cells were treated with complex 2a (1 mM) for 24 h in the absence or presence of different concentrations of NAC and GSH. Cell viability was measured by MTT assay. Data are presented as means±SEM of three independent experiments. *p<0.05, **p<0.005, ***p<0.001, compared with the cell viability of complex 2a alone.

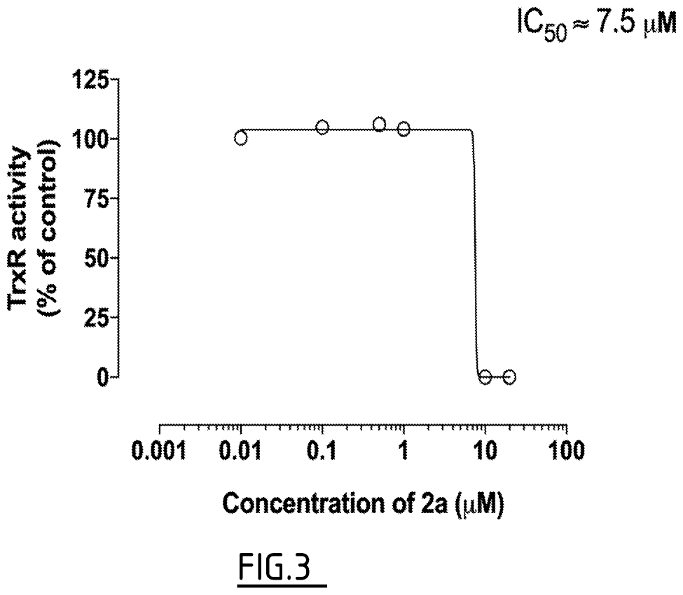

FIG. 3. IC₅₀ value of complex 2a towards isolated mammalian TrxR.

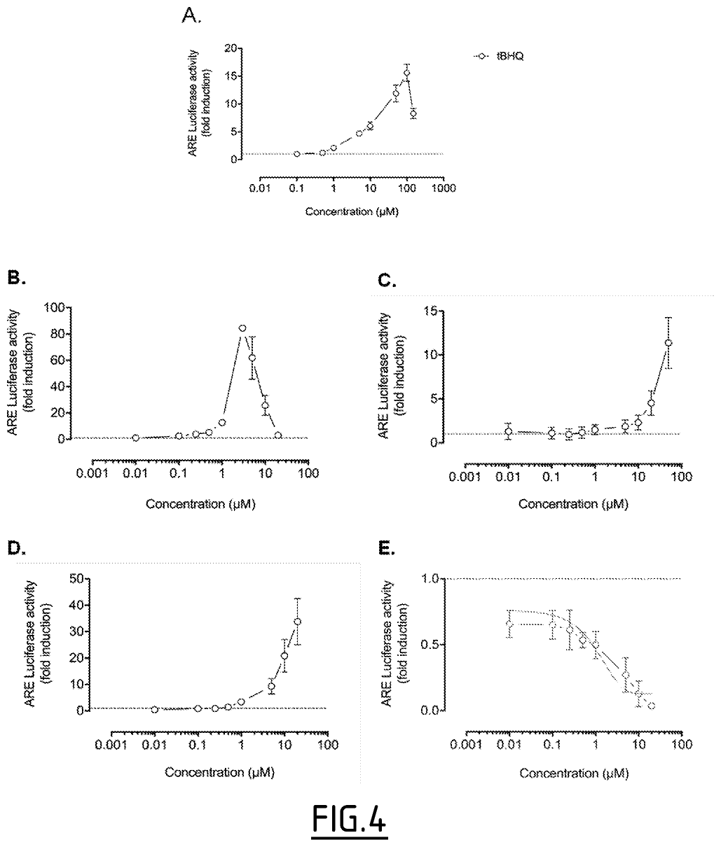

FIG. 4. NRF2 transcriptional activity.

The ARE Reporter Hep G2 cell line containing a firefly luciferase gene under the control of ARE stably integrated into Hep G2 cells was used to quantify NRF2 transcriptional activity after 16 hours of treatment with the indicated doses of the different complexes. The results are shown as fold induction of ARE luciferase reporter expression. Dashed line indicates a fold induction of 1 (values >1 mean activation and values <1 mean inhibition).

The cell line was validated for the response to the stimulation of tert-butylhydroquinone (tBHQ) according to the manufacturer's instructions (A).

Dose responses of ARE Reporter Hep G2 cells are shown to Auranofin (B), DHA (C), the compound 3 (D) and the compound 2a (E) where the “log(inhibitor) vs. Response” is represented in continuous light grey line (E).

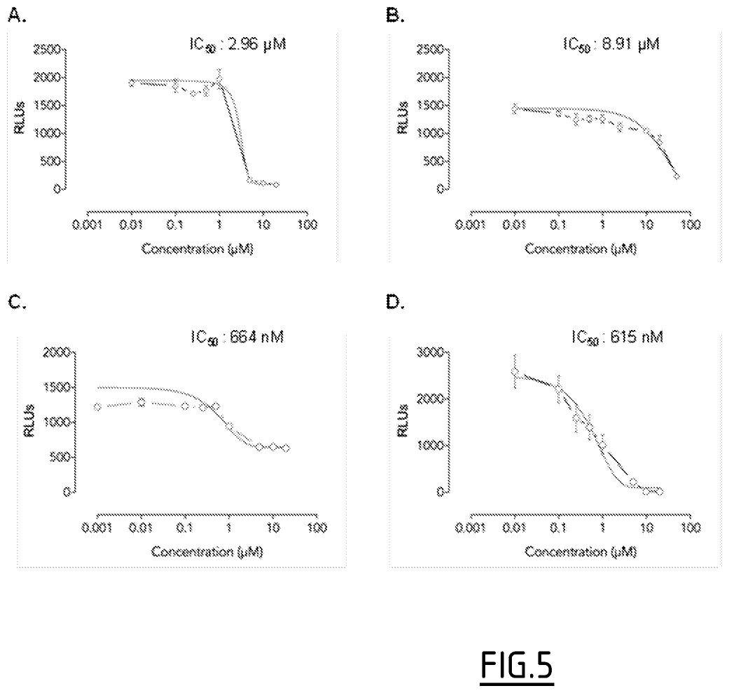

FIG. 5. NF-kB transcriptional activity.

The NF-kB Reporter (Luc)—A549 Stable Cell Line was used to quantify the inhibitory effects of the indicated doses of the molecules of the invention on transcriptional activity of NF-kB activated by 1 ng/ml TNFα (7 hours of treatment).

Luminescence was read using a luminometer and readings were normalized to wells that only contain media to obtain the Relative Luminescence Units (RLUs).

Error bar=standard deviation (SD).

Dose responses of NF-kB Reporter—A549 cells activated by TNFα to Auranofin (A), DHA (B), the compound 3 (C) and the compound 2a (D) where the “log(inhibitor) vs. Response” is represented in continuous light grey lines (A to 0).

### EXAMPLE

1. Materials

All complexation reactions were performed under an inert atmosphere of dry nitrogen by using standard vacuum line and Schlenk tube techniques. Reactions involving silver compounds were performed with the exclusion of light. CH₃CN was dried over CaH₂ and subsequently distilled. 10β-(20-Bromopropoxy)dihydroartemisinin (DHA-C3) was synthetized according a modified literature procedure.^{[1]} All other reagents were used as received from commercial suppliers.

Human prostate cancer PC-3 and lung carcinoma A549 cell lines were obtained from DSMZ (Braunschweig, Germany). Human bladder cancer T24, human osteosarcoma U-2 OS, human breast cancer MCF-7, human hepatocarcinoma HepG2 cells, human normal epithelial prostate RPWE-1, human chronic myeloid leukemia LAMA, mouse osteoblasts MC3T3 and murine fibroblasts NIH3T3 were from ATCC-LGC Standards (Molsheim, France). All the cell culture medium, fetal bovine serum (FBS) and phosphate-buffered saline (PBS) were purchased from Thermo Fisher Scientific. N-Acetyl-L-cysteine (NAC), reduced Glutathione (GSH) and 3-(4,5-dimethylthiazol-2-yl)-2,5-diphenyltetrazolium bromide (MTT) were obtained from Sigma-Aldrich.

2. Instrumentation

¹H (300 or 400 MHz) and ¹³C NMR spectra (75 or 101 MHz) and 2D experiments were recorded at 298 K on Bruker AV300, Bruker AV400 or Bruker Avance 500 spectrometers in CDCl₃ as solvent. All chemical shifts for ¹H and ¹³C are relative to TMS using ¹H (residual) or ¹³C chemical shifts of the solvent as a secondary standard. All the ¹H and ¹³C signals were assigned based on chemical shifts, spin-spin coupling constants, splitting patterns and signal intensities, and by using ¹H-¹H COSY45, ¹H-¹³C HMBC and ¹H-¹³C HSQC/HMQC, experiments for complexes 2a-2c. Gradient-enhanced ¹H COSY45 was realised included 2 scans per increment. ¹H-¹³C correlation spectra using a gradient-enhanced HSQC/HMQC sequence (delay was optimised for ¹J_{CH} of 145 Hz) was obtained with 2 scans per increment. Gradient-enhanced HMBC experiment was performed allowing 62.5 ms for long-range coupling evolution (8 scans were accumulated). Typically, 1024 t2 data points were collected for 256 t1 increments. High Resolution Mass Spectrometry (HRMS) analysis were performed with a Xevo G2 QTOF Waters spectrometer using electrospray ionization (ESI) by the “Service de Spectrométrie de Masse de Chimie UPS-CNRS (Toulouse)”. Elemental analyses were carried out by the “Service de Microanalyse du Laboratoire de Chimie de Coordination (Toulouse)”. The absorbance for MTT assay was measured using a Promega E7031 microplate reader.

3. Synthesis of Proligands 1a-c and Complexes 2a-c

The general scheme for preparing the complexes 2a-c is the following:

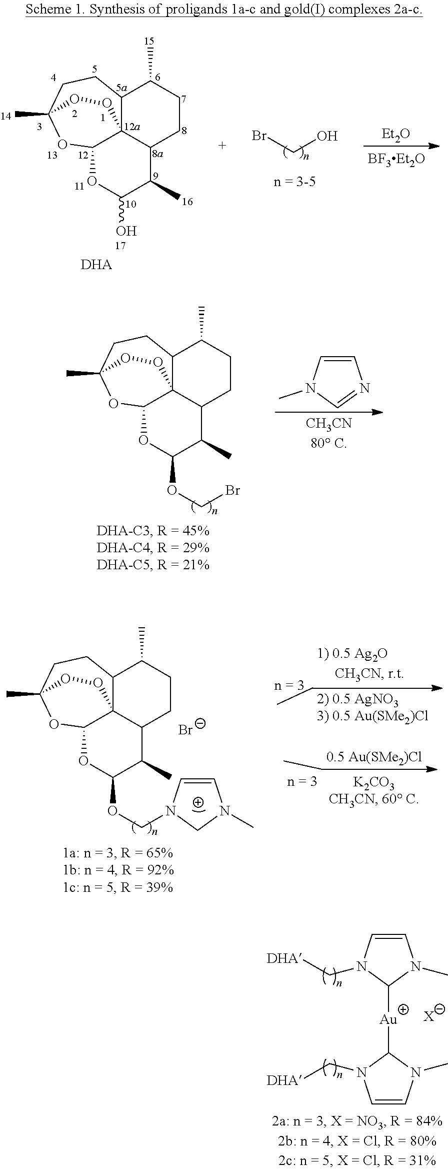

Complexes 2a-c are compounds of formula (I) according to the invention.

In order to fuse DHA and NHCs precursors the inventors used aliphatic linkers of different lengths C3 to C5. The synthesis (Scheme 1) starts with the formation of an ether, by reacting commercially DHA with a bromoalcohol in the presence of boron trifluoride etherate catalyst according to the procedure described by Haynes for the C3-derivative, leading to the single β-isomer DHA-C3 to DHA-C5 (see reference 1). The next step was the reaction between the bromoalkyl DHA derivatives and methyl imidazole in order to obtain the corresponding carbene precursors 1a to 1c with yields ranging from 39 to 92%. The formation of the target gold complexes has been achieved by two approaches. For the C3 derivative, the convenient transmetalation route involving the mild base Ag₂O, followed by an ion exchange with AgNO₃ and subsequent addition of Au(SMe₂)Cl has been used. For the C4 and C5 derivatives, the direct metalation involving K₂CO₃ and Au(SMe₂)Cl has been applied. The gold(I) complexes 2a-c were isolated after purification by chromatography as white solids with yields of 31 to 84%. All compounds were characterized by ¹H and ¹³C NMR spectroscopy, high-resolution mass spectrometry and elemental analysis.

3.1. Synthesis of Proligands 1a-c

The following picture describes the numbering of H (¹H NMR) and C (¹³C NMR). These notations are used in the following experimental section.

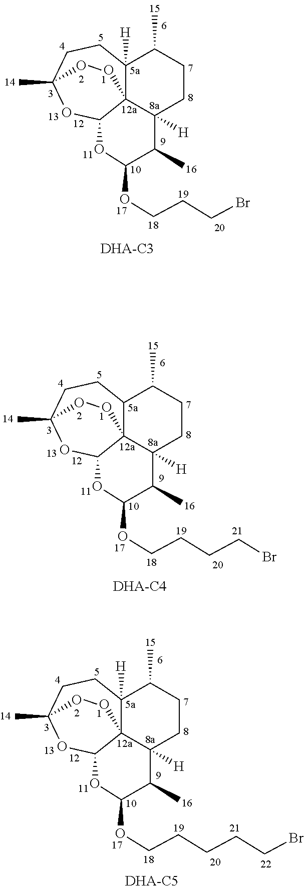 [SMILES: C[C@H]1[C@@H](OCCCBr)O[C@@H]2O[C@@]3(C)CC[C@H]4[C@H](C)CC[C@@H]1[C@@]24OO3 ; C[C@H]1[C@@H](OCCCCBr)O[C@@H]2O[C@@]3(C)CCC4[C@H](C)CC[C@@H]1[C@]42OO3 ; C[C@H]1[C@@H](OCCCCCBr)O[C@@H]2O[C@@]3(C)CC[C@H]4[C@H](C)CC[C@@H]1[C@@]24OO3]

10β-(20-Bromopropoxy)dihydroartemisinin (DHA-C3)^{[1]}

Under a nitrogen atmosphere, dihydroartemisinin (DHA) (2 g, 7.0 mmol) was dissolved in 200 mL Et₂O. 3-Bromopropan-1-ol (0.76 mL, 8.4 mmol, 1.2 eq.) and BF₃.Et₂O (6 drops) were added and the reaction mixture was stirred for 4 h at room temperature. Then the solution was treated with a saturated solution of NaHCO₃ and the product was extracted with Et₂O (3×20 mL). The combined organic phases were dried over Na₂CO₃, filtered and the solvent was evaporated to dryness. The crude product was purified by column chromatography on silica using hexane-ethyl acetate as eluent (100/0 to 100/20) to give a white solid (1.277 g, 45% yield). ¹H NMR (400 MHz, CDCl₃): δ=5.44 (s, 1H, H12), 4.82 (d, J=3.4 Hz, 1H, H10), 4.04-3.97 (m, 1H, H18), 3.54-3.47 (m, 3H, H18, H20), 2.70-2.60 (m, 1H, H9), 2.43-2.33 (m, 1H, H4), 2.15-2.06 (m, 2H, H19), 2.03-2.01 (m, 1H, H4), 1.94-1.85 (m, 1H, H5), 1.80-1.72 (m, 2H, H8), 1.68-1.62 (m, 1H, H7), 1.54-1.50 (m, 1H, H8a), 1.49-1.47 (m, 1H, H5), 1.46 (s, 3H, H14), 1.37-1.30 (m, 1H, H6), 1.29-1.23 (m, 1H, H5a), 0.97 (d, J=6.3 Hz, 3H, H15), 0.94-0.89 (m, 1H, H7), 0.92 (d, J=7.4 Hz, 3H, H16). ¹³C NMR (75 MHz, CDCl₃): δ=104.05 (1C, C3), 102.07 (1C, C10), 87.89 (1C, C12), 80.99 (1C, C12a), 65.66 (1C, C18), 52.56 (1C, C5a), 44.36 (1C, C8a), 37.42 (1C, C6), 36.40 (1C, C4), 34.62 (1C, C7), 32.52 (1C, C19), 30.86 (1C, C9), 30.57 (1C, C20), 26.16 (1C, C14), 24.65-24.49 (2C, C5, C8), 20.35 (1C, C15), 12.96 (1C, C16).

### 10β-(21-Bromobutoxy)dihydroartemisinin (DHA-C4)

Under a nitrogen atmosphere, dihydroartemisinin (DHA, 500 mg, 1.76 mmol) was dissolved in 200 mL Et₂O. 4-Bromobutan-1-ol (398 mg, 2.6 mmol, 1.48 eq.) and BF₃.Et₂O (6 drops) were added and the reaction mixture was stirred for 4 h at room temperature. Then the solution was treated with a saturated solution of NaHCO₃ and the product was extracted with Et₂O (3×20 mL). The combined organic phases were dried over Na₂CO₃, filtered and the solvent was evaporated to dryness. The crude product was purified by column chromatography on silica using hexane-ethyl acetate as eluent (100/0 to 100/20) to give a white solid (220.3 mg, 29% yield). Anal. Calcd. for C₁₉H₃₁BrO₅: C, 54.42; H, 7.45. Found: C, 54.36; H, 7.38. ¹H NMR (400 MHz, CDCl₃): δ=5.40 (s, 1H, H12), 4.80 (d, J=3.2 Hz, 1H, H10), 3.9-3.88 (m, 1H, H18), 3.50-3.36 (m, 3H, H18, H21), 2.71-2.59 (m, 1H, H9), 2.45-2.32 (m, 1H, H4), 2.08-2.03 (m, 1H, H4), 1.99-1.86 (m, 3H, H19, H5), 1.83-1.74 (m, 4H, H8, H20), 1.70-1.63 (m, 1H, H7), 1.56-1.53 (m, 1H, H8a), 1.51-1.49 (m, 1H, H5), 1.46 (s, 3H, H14), 1.38-1.32 (m, 1H, H6), 1.30-1.26 (m, 1H, H5a), 0.98 (d, J=6.3 Hz, 3H, H15), 0.97-0.95 (m, 1H, H7), 0.92 (d, J=7.4 Hz, 3H, H16). ¹³C NMR (101 MHz, CDCl₃): δ=104.10 (1C, C3), 102.02 (1C, C10), 87.92 (1C, C12), 81.10 (1C, C12a), 67.44 (1C, C18), 52.58 (1C, C5a), 44.42 (1C, C8a), 37.49 (1C, C6), 36.44 (1C, C4), 34.64 (1C, C7), 33.63 (1C, C21), 30.90 (1C, C9), 29.83 (1C, C19), 28.34 (1C, C20), 26.22 (1C, C14), 24.69-24.51 (2C, C5, C8), 20.37 (1C, C15), 13.04 (1C, C16). HRMS (ES⁺): calcd. for C₁₉H₃₁BrNaO₅ m/z=441.1253; found 441.1251.

### 10β-(22-Bromopentoxy)dihydroartemisinin (DHA-C5)

Under a nitrogen atmosphere, dihydroartemisinin (DHA, 1 g, 3.5 mmol) was dissolved in 200 mL Et₂O. 5-Bromopentan-1-ol (601 mg, 3.6 mmol, 1.03 eq.) and BF₃.Et₂O (6 drops) were added and the reaction mixture was stirred for 4 h at room temperature. Then the solution was treated with a saturated solution of NaHCO₃ and the product was extracted with Et₂O (3×20 mL). The combined organic phases were dried over Na₂CO₃, filtered and the solvent was evaporated to dryness. The crude product was purified by column chromatography on silica using hexane-ethyl acetate as eluent (100/0 to 100/20) to give a white solid (314.4 mg, 21% yield). Anal. Calcd. for C₂₀H₃₃BrO₅: C, 55.43; H, 7.68. Found: C, 55.49; H, 7.82. ¹H NMR (400 MHz, CDCl₃): δ=5.41 (s, 1H, H12), 4.79 (d, J=3.2 Hz, 1H, H10), 3.87 (dt, J=9.8, 6.2 Hz, 1H, H18), 3.45-3.37 (m, 3H, H18, H22), 2.66-2.61 (m, 1H, H9), 2.39 (ddd, J=14.5, 13.4, 3.9 Hz, 1H, H4), 2.08-2.02 (m, 1H, H4), 1.94-1.87 (3H, H5, H21), 1.82-1.77 (m, 1H, H7), 1.67-1.48 (m, 8H, H5, H8, H8a, H19, H20), 1.50 (s, 3H, H14), 1.40-1.32 (m, 1H, H6), 1.29-1.22 (m, 1H, H5a), 0.97 (d, J=6.3 Hz, 3H, H15), 0.98-0.91 (m, 1H, H7), 0.92 (d, J=7.4 Hz, 3H, H16). ¹³C NMR (101 MHz, CDCl₃): δ=104.05 (1C, C3), 101.99 (1C, C10), 87.92 (1C, C12), 81.12 (1C, C12a), 68.01 (1C, C18), 52.58 (1C, C5a), 44.45 (1C, C8a), 37.46 (1C, C6), 36.44 (1C, C4), 34.66 (1C, C7), 33.81 (1C, C22), 32.41 (1C, C21), 30.92 (1C, C9), 28.82 (1C, C19), 26.21 (1C, C14), 24.96 (1C, C20), 24.68-24.48 (2C, C5, C8), 20.36 (1C, C15), 13.04 (1C, C16). HRMS (ES⁺): calcd. for C₂₀H₃₃BrNaO₅ m/z=455.1409; found 455.1409.

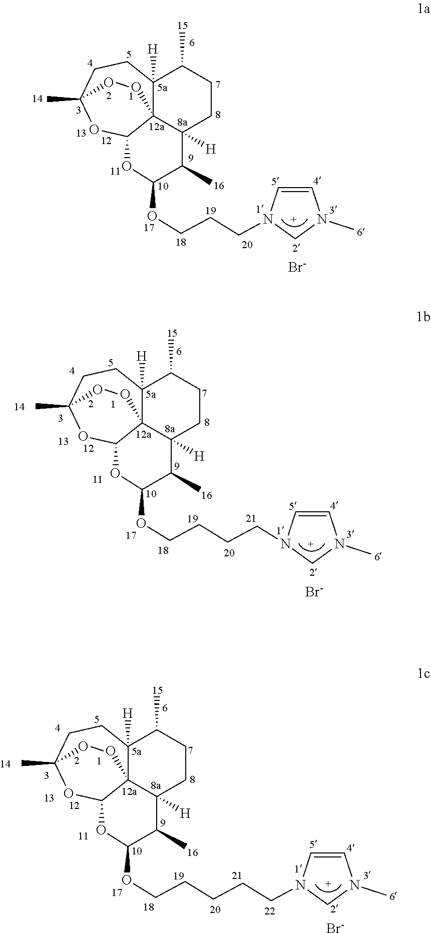

### 3′-methyl-1′-[10β-(20-propoxy)dihydroartemisin]1H-imidazol-3-ium bromide (1a)

To a stirred solution of DHA-C3 (304 mg, 0.75 mmol) in CH₃ON (6 mL) under reflux, 1-methylimidazole (60 μL, 0.75 mmol) was added and the reaction mixture was stirred 3 days under reflux. The solvent was evaporated under reduced pressure and the viscous residue was purified by chromatography on silica with CH₂Cl₂-MeOH as eluent (1/0.1 to 1/0.25) to afford a white solid (238 mg, 65% yield). Anal. Calcd. for C₂₂H₃₅BrN₂O₅: C, 54.21; H, 7.24; N, 5.75. Found C, 54.12; H, 7.26; N, 5.68. ¹H NMR (400 MHz, CDCl₃): δ=10.44 (s, 1H, H2′), 7.48 (s, 1H, H4′), 7.38 (s, 1H, H5′), 5.38 (s, 1H, H12), 4.79 (d, J=3.6 Hz, 1H, H10), 4.44 (t, J=7.2 Hz, 2H, H20), 4.14 (s, 3H, H6′), 3.92-3.86 (m, 1H, H18), 3.53-3.47 (m, 1H, H18), 2.68-2.61 (m, 1H, H9), 2.37 (ddd, J=14.6, 13.4, 4.0 Hz, 1H, H4), 2.29-2.22 (m, 2H, H19), 2.04 (ddd, J=14.6, 4.9, 2.9 Hz, 1H, H4), 1.93-1.86 (m, 1H, H5), 1.81-175 (m, 1H, H7), 1.72-1.61 (m, 2H, H8), 1.51-1.44 (m, 2H, H5, H8a), 1.42 (s, 3H, H14), 1.38-1.31 (m, 1H, H6), 1.29-1.23 (m, 1H, H5a), 0.97 (d, J=6.3 Hz, 3H, H15), 0.95-0.89 (m, 1H, H7), 0.92 (d, J=7.4 Hz, 3H, H16). ¹³C NMR (101 MHz, CDCl₃): δ=137.90 (1C, C2′), 123.37 (1C, C4′), 122.15 (1C, C5′), 104.23 (1C, C3), 102.20 (1C, C10), 87.96 (1C, C12), 80.92 (1C, C12a), 64.60 (1C, C18), 52.42 (1C, C5a), 47.48 (1C, C20), 44.16 (1C, C8a), 37.45 (1C, C6′), 36.90 (1C, C6), 36.33 (1C, C4), 34.45 (1C, C7), 30.77 (1C, C9), 30.60 (1C, C19), 26.10 (1C, C14), 24.63-24.57 (2C, C5, C8), 20.29 (1C, C15), 13.13 (1C, C16). HRMS (ES⁺): calcd. for C₂₂H₃₅N₂O₅ m/z=407.2545; found 407.2546.

### 3′-methyl-1′-[10β-(21-butoxy)dihydroartemisin]1H-imidazol-3-ium bromide (1 b)

To a stirred solution of DHA-C4 (75 mg, 0.18 mmol) in CH₃CN (3 mL) under reflux, 1-methylimidazole (57 μL, 0.72 mmol, 4 eq.) was added and the reaction mixture was stirred 5 days under reflux. The solvent was evaporated under reduced pressure and the viscous residue was purified by chromatography on silica with CH₂Cl₂-MeOH as eluent (1/0.1 to 1/0.25) to afford a white solid (83 mg, 92% yield). Anal. Calcd. for C₂₃H₃₇BrN₂O₅: C, 55.09; H, 7.44; N, 5.59. Found C, 55.12; H, 7.56; N, 5.58. ¹H NMR (400 MHz, CDCl₃): δ=10.38 (s, 1H, H2′), 7.55 (t, 1H, J=1.8 Hz, H4′), 7.42 (t, 1H, J=1.8 Hz, H5′), 5.34 (s, 1H, H12), 4.75 (d, J=3.4 Hz, 1H, H10), 4.37 (t, J=7.4 Hz, 2H, H21), 4.10 (s, 3H, H6′), 3.83 (dt, 1H, J=9.9, 6.0 Hz, H18), 3.40 (dt, 1H, J=9.9, 6.4 Hz, H18), 2.62-2.52 (m, 1H, H9), 2.37-2.29 (m, 1H, H4), 2.04-1.83 (m, 3H, H4, H20), 1.90-1.83 (m, 1H, H5), 1.73-1.59 (m, 5H, H7, H8, H19), 1.51-1.41 (m, 2H, H8a, H5), 1.39 (s, 3H, H14), 1.35-1.27 (m, 1H, H6), 1.25-1.17 (m, 1H, H5a), 0.94 (d, J=6.3 Hz, 3H, H15), 0.91-0.83 (m, 1H, H7), 0.87 (d, J=7.3 Hz, 3H, H16). ¹³C NMR (101 MHz, CDCl₃): δ=137.95 (1C, C2′), 123.41 (1C, C4′), 121.59 (1C, C5′), 104.13 (1C, C3), 102.12 (1C, C10), 87.91 (1C, C12), 81.03 (1C, C12a), 67.43 (1C, C18), 52.49 (1C, C5a), 49.88 (1C, C21), 44.29 (1C, C8a), 37.43 (1C, C6′), 36.82 (1C, C6), 36.38 (1C, C4), 34.51 (1C, C7), 30.84 (1C, C9), 27.28-26.44 (2C, C19, C20), 26.17 (1C, C14), 24.65-24.52 (2C, C5, C8), 20.32 (1C, C15), 13.09 (1C, C16). HRMS (ES⁺): calcd. for C₂₃H₃₇N₂O₅ m/z=421.2702; found 421.2704.

### 3′-methyl-1′-[10β-(22-pentoxy)dihydroartemisin]1H-imidazol-3-ium bromide (1c)

To a stirred solution of DHA-C5 (189 mg, 0.45 mmol) in CH₃CN (6 mL) under reflux, 1-methylimidazole (143 μL, 1.80 mmol, 4 eq.) was added and the reaction mixture was stirred 3 days under reflux. The solvent was evaporated under reduced pressure and the viscous residue was purified by chromatography on silica with CH₂Cl₂-MeOH as eluent (1/0.1 to 1/0.25) to afford a white solid (91 mg, 39% yield). Anal. Calcd. for C₂₄H₃₉BrN₂O₅: C, 55.92; H, 7.63; N, 5.43. Found C, 55.86; H, 7.56; N, 5.38. ¹H NMR (400 MHz, CDCl₃): δ=10.40 (s, 1H, H2′), 7.54 (t, 1H, J=1.8 Hz, H4′), 7.43 (t, 1H, J=1.8 Hz, H5′), 5.34 (s, 1H, H12), 4.73 (d, J=3.6 Hz, 1H, H10), 4.33 (t, J=7.5 Hz, 2H, H22), 4.12 (s, 3H, H6′), 3.79 (dt, 1H, J=9.8, 6.3 Hz, H18), 3.45 (dt, 1H, J=9.8, 6.5 Hz, H18), 2.62-2.54 (m, 1H, H9), 2.38-2.30 (m, 1H, H4), 2.04-1.92 (m, 3H, H4, H21), 1.89-1.83 (m, 1H, H5), 1.74-1.58 (m, 5H, H7, H8, H19), 1.53-1.37 (m, 4H, H5, H8a, H20), 1.40 (s, 3H, H14), 1.35-1.29 (m, 1H, H6), 1.27-1.18 (m, 1H, H5a), 0.94 (d, J=6.3 Hz, 3H, H15), 0.90-0.83 (m, 1H, H7), 0.86 (d, J=7.3 Hz, 3H, H16). ¹³C NMR (101 MHz, CDCl₃): δ=137.60 (1C, C2′), 123.51 (1C, C4′), 121.89 (1C, C5′), 104.07 (1C, C3), 101.99 (1C, C10), 87.88 (1C, C12), 81.09 (1C, C12a), 67.86 (1C, C18), 52.52 (1C, C5a), 50.00 (1C, C22), 44.37 (1C, C8a), 37.44 (1C, C6′), 36.77 (1C, C6), 36.40 (1C, C4), 34.56 (1C, C7), 30.88 (1C, C9), 30.06-29.01 (2C, C19, C21), 26.19 (1C, C14), 24.66-24.48 (2C, C5, C8), 23.00 (1C, C20), 20.35 (1C, C15), 13.05 (1C, C16). HRMS (ES⁺): calcd. for C₂₄H₃₉N₂O₅ m/z=435.2859; found 435.2861.

3.2. Synthesis of Complexes 2a-c

Complex 2a

In a Schlenk tube, 1a (102 mg, 0.21 mmol) and Ag₂O (25 mg, 0.11 mmol) were dissolved in CH₃CN (3 mL) under a nitrogen atmosphere and protection of the light and stirred overnight at room temperature. A solution of AgNO₃ (19 mg, 0.11 mmol) in 2 mL of CH₃CN was added and the mixture was stirred for 2 h. Finally, Au(SMe₂)Cl (32 mg, 0.11 mmol) was added and after stirring for 1 h at room temperature, the solution was filtered through a pad of celite and the solvent removed under reduced pressure to afford a white solid (95 mg, 84% yield). Crystals suitable for X-ray diffraction analysis were obtained by slow diffusion of Et₂O in a CH3CN solution of this complex. Anal. Calcd. For C₄₄H₆₈AuN₅O₁₃: C, 49.30; H, 6.39; N, 6.53. Found C, 49.32; H, 6.45; N, 5.49. ¹H NMR (400 MHz, CDCl₃): 57.26 (d, J=1.8 Hz, 1H, H4′), 7.16 (d, J=1.9 Hz, 1H, H5′), 5.37 (s, 1H, H12), 4.79 (d, J=3.5 Hz, 1H, H10), 4.28 (t, J=7.0 Hz, 2H, H20), 3.95 (s, 3H, H6′), 3.91-3.89 (m, 1H, H18), 3.46-3.43 (m, 1H, H18), 2.64-2.62 (m, 1H, H9), 2.37-2.34 (m, 1H, H4), 2.19-2.16 (m, 2H, H19), 2.04-2.02 (m, 1H, H4), 1.89-1.87 (m, 1H, H5), 1.73-1.71 (m, 2H, H8), 1.61-1.59 (m, 1H, H7), 1.48-1.46 (m, 1H, H8a), 1.46-1.43 (m, 1H, H5), 1.42 (s, 3H, H14), 1.32-1.30 (m, 1H, H6), 1.25-1.20 (m, 1H, H5a), 0.96 (d, J=6.2 Hz, 3H, H15), 0.95-0.92 (m, 1H, H7), 0.91 (d, J=7.4 Hz, 3H, H16). ¹³C NMR (101 MHz, CDCl₃): δ 183.71 (1C, C2′), 123.26 (1C, C4′), 121.84 (1C, C5′), 104.19 (1C, C3), 101.98 (1C, C10), 87.93 (1C, C12), 80.89 (1C, C12a), 64.81 (1C, C18), 52.46 (1C, C5a), 48.50 (1C, C20), 44.22 (1C, C8a), 38.21 (1C, C6′), 37.52 (1C, C6), 36.35 (1C, C4), 34.50 (1C, C7), 31.70 (1C, C19), 30.75 (1C, C9), 26.11 (1C, C14), 24.66-24.54 (2C, C5, C8), 20.31 (1C, C15), 13.10 (1C, C16) HRMS (ES⁺): calcd. for C₄₄H₆₈AuN₄O₁₀ m/z 1009.4601; found 1009.4591.

Complex 2b

Under a nitrogen atmosphere, potassium carbonate (27.7 mg, 0.20 mmol) was added to a mixture of 1 b (80 mg, 0.16 mmol) and Au(SMe₂)Cl (26.5 mg, 0.09 mmol) in dry CH3CN (5 mL). The mixture was then heated to 60° C. and stirred for 2 h. After cooling to room temperature, the solution was filtered through a pad of celite and the solvent removed under reduced pressure. The complex was purified by preparative chromatography on silica plate with CH₂Cl₂-MeOH as eluent (100/8) to afford a white solid (68.7 mg, 80% yield). Anal. Calcd. For C₄₆H₇₂AuClN₄O₁₀. C, 51.47; H, 6.76; N, 5.22. Found C, 51.39; H, 6.68; N, 5.18. ¹H NMR (400 MHz, CDCl₃): δ 7.37 (d, J=1.9 Hz, 1H, H4′), 7.22 (d, J=1.9 Hz, 1H, H5′), 5.33 (s, 1H, H12), 4.74 (d, J=3.2 Hz, 1H, H10), 4.25 (t, J=7.0 Hz, 2H, H21), 3.97 (s, 3H, H6′), 3.86-3.80 (m, 1H, H18), 3.42-3.37 (m, 1H, H18), 2.61-2.57 (m, 1H, H9), 2.38-2.30 (m, 1H, H4), 2.04-1.84 (m, 4H, H4, H5, H20), 1.76-1.58 (m, 5H, H7, H8, H19), 1.46-1.37 (m, 2H, H5, H8a), 1.40 (s, 3H, H14), 1.30-1.19 (m, 2H, H5a, H6), 0.94 (d, J=5.9 Hz, 3H, H15), 0.95-0.84 (m, 1H, H7), 0.86 (d, J=7.3 Hz, 3H, H16). ¹³C NMR (101 MHz, CDCl₃): δ 183.57 (1C, C2′), 123.45 (1C, C4′), 121.53 (1C, C5′), 104.10 (1C, C3), 101.99 (1C, C10), 87.87 (1C, C12), 80.97 (1C, C12a), 67.50 (1C, C18), 52.40 (1C, C5a), 51.04 (1C, C21), 44.27 (1C, C8a), 38.42 (1C, C6′), 37.49 (1C, C6), 36.35 (1C, C4), 34.53 (1C, C7), 30.79 (1C, C9), 28.34 (1C, C20), 26.73, (1C, C19), 26.16 (1C, C14), 24.65-24.48 (2C, C5, C8), 20.35 (1C, C15), 13.08 (1C, C16) HRMS (ES⁺): calcd. for C₄₆H₇₂AuN₄O₁₀ m/z 1037.4914; found 1037.4929.

Complex 2c

Under a nitrogen atmosphere, potassium carbonate (38.7 mg, 0.28 mmol) was added to a mixture of 1c (118.5 mg, 0.23 mmol) and Au(SMe₂)Cl (35.3 mg, 0.12 mmol) in dry CH₃CN (5 mL). The mixture was then heated to 60° C. and stirred for 2 h. After cooling to room temperature, the solution was filtered through a pad of celite and the solvent removed under reduced pressure. The complex was purified by preparative chromatography on silica plate with CH₂Cl₂-MeOH as eluent (100/8) to afford a white solid (39.3 mg, 31% yield). Anal. Calcd. For C₄₈H₇₆AuClN₄O₁₀. C, 52.34; H, 6.95; N, 5.09. Found C, 52.39; H, 6.98; N, 5.08. ¹H NMR (300 MHz, CDCl₃): δ 7.32 (d, J=1.9 Hz, 1H, H4′), 7.22 (d, J=1.9 Hz, 1H, H5′), 5.36 (s, 1H, H12), 4.75 (d, J=3.6 Hz, 1H, H10), 4.25 (t, J=7.0 Hz, 2H, H22), 3.99 (s, 3H, H6′), 3.88-3.78 (m, 1H, H18), 3.41-3.33 (m, 1H, H18), 2.63-2.58 (m, 1H, H9), 2.43-2.32 (m, 1H, H4), 2.08-1.97 (m, 4H, H4, H5, H21), 1.78-1.59 (m, 5H, H7, H8, H19), 1.53-1.38 (m, 4H, H5, H8a, H20), 1.43 (s, 3H, H14), 1.30-1.24 (m, 2H, H5a, H6), 0.97 (d, J=6.1 Hz, 3H, H15), 0.97-0.84 (m, 1H, H7), 0.87 (d, J=7.4 Hz, 3H, H16). ¹³C NMR (101 MHz, CDCl₃): 183.59 (1C, C2′), 123.28 (1C, C4′), 121.65 (1C, C5′), 104.08 (1C, C3), 101.91 (1C, C10), 87.86 (1C, C12), 81.01 (1C, C12a), 67.85 (1C, C18), 52.49 (1C, C5a), 51.22 (1C, C22), 44.32 (1C, C8a), 38.38 (1C, C6′), 37.48 (1C, C6), 36.37 (1C, C4), 34.58 (1C, C7), 31.22 (1C, C21), 30.82 (1C, C9), 29.13 (1C, C19), 26.18 (1C, C14), 24.66-24.46 (2C, C5, C8), 23.23 (1C, C20), 20.37 (1C, C15), 13.02 (1C, C16) HRMS (ES⁺): calcd. for C₄₈H₇₆AuN₄O₁₀ m/z 1065.5221; found 1065.5226.

¹³C NMR spectroscopy unequivocally evidences the formation of the cationic gold(I) complexes with resonance of the carbenic carbons located at 183.6-183.7 ppm. HRMS spectra of 2a-c exhibit the classical peak for the cationic fragment [M-X⁻]⁺ and elemental analysis correspond to the general [AuL₂][X] formula.

4. Crystallographic Data for 2a

Single crystals suitable for X-ray structure analysis have been obtained by gas phase diffusion from diethyl ether to a saturated solution of 2a in acetonitrile. In the solid state the gold(I) shows the typical linear coordination stabilized by two NHC ligands. The NHC planes are crossed around the C—Au—C axis with torsion angles from 116° to 138°. It is remarkable that the bulky DHA-derivative groups are on the same side of the central bisNHC gold motif.

This is due to an aurophilic interaction leading to a dimeric form of the complex with Au—Au distance of 345.0 pm.

All data were collected at low temperature using oil-coated shock-cooled crystals on a Bruker-AXS APEX II diffractometer with MoKa radiation (λ=0.71073 Å). The structure was solved by direct methods^{[2]} and all non hydrogen atoms were refined anisotropically using the least-squares method on F².^{[3]} The absolute structure parameters have been refined using the Flack-method.^{[4]}

Complex 2a: C₄₄H₆₈AuN₅O_{13.12}, Mr=1074.0, crystal size=0.40×0.30×0.05 mm³, orthorhombic, space group I222, a=10.644(2) Å, b=19.073(3) Å, c=48.320(8) Å, V=9809(3) Å³, Z=8, 59886 reflections collected, 6963 unique reflections (Rᵢₙₜ=0.1151), R1=0.0568, wR2=0.1300 [I>2σ(I)], R1=0.1309, wR2=0.1727 (all data), absolute structure factor x=−0.021(8), residual electron density=3.308 e Å⁻³.

5. Cell Culture and Cell Viability Assay (MTT Assay)

PC-3 human prostate cancer cells, and LAMA chronic myeloid leukemia were cultured in RPMI 1640 containing 10% fetal bovine serum and 1% antibiotics (100 U/mL penicillin and 100 μg/mL streotimycin) at 37° C. in 5% CO₂ humidified incubators. HL60 chronic myeloid leukemia were cultured in RPMI 1640 containing 15% fetal bovine serum and 1% antibiotics (100 U/mL penicillin and 100 μg/mL streotimycin) at 37° C. in 5% CO₂ humidified incubators. HepG-2 human liver cancer cells were cultured in EMEM containing 10% of FBS, 1% of nonessential amino acids, 1 mM of Na-pyruvate, 1% of PenStrep antibiotics and 600 μg/mL of Geneticin. A549 human lung carcinoma cells, T24 human bladder carcinoma cells, MCF-7 human breast adenocarcinoma cells, U-2OS human osteosarcoma and NIH3T3 murine fibroblast cells were cultured in DMEM medium containing 10% fetal bovine serum and 1% antibiotics at 37° C. in 5% CO₂ humidified incubators. MC3T3 mouse osteoblast cells were cultured in MEM medium containing 10% fetal bovine serum and 1% antibiotics at 37° C. in 5% CO₂ humidified incubators. RWPE-1 human prostate normal cells were cultured in K-SFM medium containing 0.05 mg/ml bovine pituitary extract (BPE) and 5 ng/ml epidermal growth factor (EGF) at 37° C. in 5% CO₂ humidified incubators. The MTT reagent (3-(4,5-dimethylthiazol-2-yl)-2,5-diphenyltetrazolium bromide) was used to determine cell death as originally described by Mosmaneland modified by Cuvillier et al.^{[6]} Briefly, cells were seeded 5,000 to 10,000 cells/well in 24-well plates depending on the cell type and allowed to attach overnight. All of the complexes were dissolved in DMSO. The concentration of the complexes was calculated according to the elemental composition of the complexes determined by the elemental analyses. Media in the presence of the tested complexes were added and serially diluted to various concentrations (from 5 μM to 0.01 μM). Next to these concentrations, for DHA 50, 20 and 10 μM and for the cationic bisNHC gold(I) complex 3 0.001 μM have been used. The maximum concentration of DMSO in media did not exceed 0.5% (v/v). After 72 h of treatment, cells were incubated at 37° C. and 5% CO₂ with 25 μL MTT solution (5 mg/mL; Sigma-Aldrich) in 24-well plates for approximately 3 to 4 h. After solubilization with 500 μL of lysis buffer (DMSO), formazan was quantified by spectrophotometry with a microplate reader at 570 nm absorbance. The GI₅₀ values corresponding to the concentration that caused 50% inhibition of cell proliferation were calculated from dose-response curves obtained by nonlinear regression analysis (Prism 8, Graphpad Software). All the results were calculated from data obtained in three independent experiments.

Precursors 1a-c and complexes 2a-c were evaluated for their in vitro cytotoxic abilities against PC-3 prostate cancer cell line and three non-cancer cell lines (fibroblast NIH3T3, osteoblast MC3T3 and epithelial prostate RPWE-1) (Table 1 below). Interestingly, the imidazolium salts 1a-c showed no cytotoxic effects (GI₅₀>20 μM), while complexes 2a-c exhibited strong antiproliferative activities with GI₅₀ values between 20 nM and 70 nM. The selectivity indexes (SI=G150 (non-cancer cell line)/G150 (cancer cell line)) of 2a-c gave nearly the same values concerning NIH3T3 cells ranging from 15.5 to 16.7, while RPWE-1 cells gave a more differentiated result with the highest SI=6.9 for 2a. As reference drugs, Auranofin, an anti-arthritis drug currently in clinical phase I and II trials as anticancer drug, and DHA were tested. Moreover a published cationic bisNHC gold(I) complex 3, containing a methyl and a quinoline substituents^{[7]}, and a mixture of 3 and DHA (1:2) were investigated in order to evaluate the potential synergetic effect of the hybrid complexes. The structure of the cationic bisNHC gold(I) complex 3 is as follows:

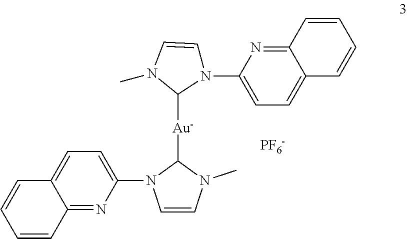

Outstandingly, complexes 2a-c displayed 16 to 55-fold and 22 to 78-fold higher potency than Auranofin and DHA on PC-3 cells, respectively. Moreover, they are 6.2 to 16.7 more selective towards cancer cells than NIH3T3 compared to the two drug references. Remarkably, complex 2a shows an SI PC-3/RPWE-1 value close to that of DHA but 69 and 35 times higher than that obtained for both gold references, Auranofin and complex 3, respectively. Complex 3 showed 10 to 35-fold lower activity than 2a-c and the mixture of 3 and DHA has an efficiency between DHA and 3 with a low selectivity. Overall, these results highlight that linking a derivative of DHA on the NHC scaffold of a bisNHC gold(I) unit led to a synergy, expressed by a high cytotoxicity combined with a high selectivity.

Due to its better selectivity, the inventors chose complex 2a for further biological investigations. 2a has been tested on a panel of seven other representative human cancer cell lines, namely A549 (lung), MCF-7 (breast), T24 (bladder), U-2 OS (bone), LAMA (leukemia), HL60 (acute myeloid leukemia) and Hep-G2 (liver) (see Table 2). As in the case of PC-3 cells the G150 values for six cancer cell lines were in the lower nM range spanning from 22 to 175 nM. Complex 2a remains extensively more effective than the control molecules used in this study on all the tested cancer cell lines, even on hepatocellular carcinoma HepG-2, which are always more difficult to treat (the commercial drug Sorafenib currently used to treat hepatocarcinoma displays an IC₅₀ of 6.4 μM on the HepG-2 cell line). Differential effects of arsenic trioxide on chemosensitization in human hepatic tumor and stellate cell lines (F. Rangwala, K. P. Williams, G. R. Smith, Z. Thomas, J. L. Allensworth, H. K. Lyerly, A. M. Diehl, M. A. Morse, G. R. Devi, BMC Cancer 2012, 12, 402), validating the concept of gold(I)-artemisinin like hybrid complexes.

TABLE 1
Cytotoxicity and selectivity of 1a-c and 2a-c
on PC-3, NIH3T3 and RWPE-1 cell lines (GI₅₀ [μM],
72 h, MTT assay).^{[a]}

| Compound | PC-3 | NIH3T3/SI^{[b]} | MC3T3/SI^{[b]} | RWPE-1/SI^{[b]} |
|---|---|---|---|---|
| 1a | >20 | >20/— | | >20/— |
| 1b | >20 | >20/— | | >20/— |
| 1c | >20 | >20/— | | 3.74/— |
| 2a | 0.070 | 1.13/16.1 | 1.62/23.1 | 0.48/6.9 |
| 2b | 0.042 | 0.70/16.7 | | 0.11/2.6 |
| 2c | 0.020 | 0.31/15.5 | | 0.098/4.9 |
| Auranofin | 1.09 | 1.10/1.0 | 1.39/1.2 | 0.084/0.1 |
| DHA | 1.56 | 3.86/2.5 | 3.55/2.3 | 9.68/6.2 |
| 3 | 0.70 | 7.98/11.4 | 17.9/25.5 | 0.112/0.2 |
| 3/DHA (1:2) | 1.09 | 2.85/2.6 | 1.76/1.6 | 0.22/0.2 |

^{[a]}The GI₅₀ values represent the concentration of compound causing 50% inhibition of cell growth. Mean of three independent experiments.
^{[b]}Selectivity index.

TABLE 2
Cytotoxicity of 2a on A549, MCF-7, T24, U2OS, LAMA, HL60 and HepG-2
lines (GI₅₀ [μM], 72 h, MTT assay).^{[a]}

| Compound | A549 | MCF-7 | T24 | U2OS | HepG-2 | LAMA | HL60 |
|---|---|---|---|---|---|---|---|
| 2a | 0.11 | 0.09 | 0.17 | 0.12 | 2.43 | 0.08 | 0.022 |
| Auranofin | 4.41 | 1.39 | 1.10 | 0.47 | 3.62 | 0.81 | 0.95 |
| DHA | 11.0 | 9.67 | 4.99 | 4.10 | 12.0 | 5.6 | 25 |
| 3 | 1.16 | 0.38 | 0.19 | 2.51 | 5.23 | 0.66 | 0.50 |
| 3/DHA | 1.99 | 0.61 | 0.32 | 1.25 | 4.71 | 0.80 | 0.47 |
| (1:2) | | | | | | | |

^{[a]}The GI₅₀ values represent the concentration of compound causing 50% inhibition of cell growth. Mean of three independent experiments.

Table 3 below also presents the IC₅₀ observed for each cell line, for the different tested molecules, which are: the compound (I) of the invention (compound 2a), auranofin, DHA, NHC-gold(I) complex 3 alone (without any artemisinin or DHA) and a mixture of NHC-gold(I) complex 3 alone and DHA alone, in a molar ratio of 1:2.

TABLE 3
IC₅₀ of 2a, auranofin, DHA, NHC-gold(I) complex alone 3 and a
mixture of 3 alone and DHA alone, in a molar ratio of 1:2 on
cancerous cells A549, MCF-7, T24, U-2 OS, PC-3, LAMA and HL60,
and on MC3T3, NIH3T3 and RWPE-1 non-cancerous cell lines.

| | | | | 3/DHA | | |
|---|---|---|---|---|---|---|
| | 2a | DHA | 3 | (ratio 1/2) | Auranofin | |
| Non tumoral cell models | | | | | | |
| MC3-T3 (osteoblasts) | 1.62 | 3.55 | 17.9 | 1.76 | 1.39 | |
| NIH3T3 (fibroblasts) | 1.13 | 3.86 | 7.98 | 2.85 | 1.10 | |
| RWPE1 (prostatic epithelial cells) | 0.48 | 9.68 | 0.112 | 0.218 | 0.084 | |
| MEAN | 1.08 | 5.70 | 8.66 | 1.61 | 0.86 | μM |
| Tumor cell models | | | | | | |
| PC-3 (prostate) | 0.070 | 1.56 | 0.,695 | 1.09 | 1.09 | |
| A549 (lung) | 0.115 | 11.04 | 1.16 | 1.99 | 4.41 | |
| U2OS (bone tumor) | 0.122 | 4.10 | 2.51 | 1.25 | 0.474 | |
| T24 (bladder) | 0.175 | 4.99 | 0.191 | 0.319 | 1.10 | |
| MCF-7 (breast) | 0.089 | 9.67 | 0.380 | 0.610 | 1.39 | |
| LAMA (chronic myeloid leukemia) | 0.079 | 5.60 | 0.662 | 0.800 | 0.809 | |
| HL60 (acute myeloid leukemia) | 0.022 | 3.25 | 0.500 | 0.471 | 0.951 | |
| MEAN of 7 tumor cell models | 0.096 | 5.74 | 0.87 | 0.93 | 1.46 | μM |

Comparison of the specificity for tumoral cells versus non tumoral cells which come from a
same tissue (e.g. prostate and bone)*

| | | | | | |
|---|---|---|---|---|---|
| Prostate: RWPE1 (normal | 6.9 | 6.2 | 0.2 | 0.2 | 0.1 |
| immortalized)/PC-3 (tumoral) | | | | | |
| Bone: MC3-T3 (normal | 13.3 | 0.9 | 7.1 | 1.4 | 2.9 |
| immortalized)/U2OS (tumoral) | | | | | |

*shows the very high specificity of the Compound 2a

6. Measurement of Intracellular Reactive Oxygen Species (ROS) and Determination of ROS Generation

The cellular ROS generation was shown by the increase of fluorescence intensity of DCF according to a previously reported protocol.^{[8]} Briefly, PC-3, A549, MCF-7 and HepG2 cells were seeded at a density of 50,000 cells/well in 24-well plates for 24 h. Cells were washed with PBS buffer and stained with DCFH-DA (final concentration 20 μM) for 45 min. Then cells were washed with PBS buffer and the culture medium without phenol red containing gold complexes were added to the cells. The fluorescence intensity of DCF (excitation/emission, 485/535 nm) was measured by fluorescence microplate reader at different time points.

For N-acetyl-cysteine (NAC) and reduced glutathione (GSH) treatment, the cells were pretreated with different concentrations of NAC and GSH (2, 5 and 10 mM) for 1 h, then gold complex 2a was added for incubation for 72 h. After that, cells were further incubated at 37° C. and 5% CO₂ with 25 μL MTT solution (5 mg/mL; Sigma-Aldrich) in 24-well plates for approximately 3 h. The cytotoxicity was determined as described above.

The results are in FIGS. 1 and 2.

7. Inhibition of Mammalian TrxR

To determine the inhibition of mammalian TrxR, an established microplate reader-based assay was performed with minor modifications.^{[9]} For this purpose, commercially available rat liver TrxR (from Sigma-Aldrich) was used and diluted with distilled water to achieve 3.5 U/mL. Complex 2a was freshly dissolved as stock solution in sterile DMSO. 25 μL Aliquot of the enzyme and either 25 μL potassium phosphate buffer (pH 7.0) containing complex 2a in graded concentrations or 25 μL buffer without the complex but DMSO (positive control) were added. The resulting solution (final DMSO concentration of 0.5% v/v) was incubated at 37° C. for 75 min with moderate shaking in a 96-well plate. To each well, 225 μL of the reaction mixture (1.0 mL reaction mixture consists of 500 μL 100 mM potassium phosphate buffer pH 7.0, 80 μL 100 mM EDTA solution pH 7.5, 20 μL 0.2% BSA, 100 μL of a 20 mM NADPH and 300 μL distilled water) was added and the reaction was initiated immediately by adding 25 μL of 20 mM DTNB solution. After thorough mixing, the formation of TNB was monitored by a microplate reader at 405 nm at 1 min intervals for 10 measurements. The increase of TNB concentration over time followed a linear tendency (r²≥0.99), and the enzymatic activities were calculated as the slopes (increase in absorbance per second). Non-interference with the assay components was confirmed by a negative control experiment using an enzyme-free test compound. 1050 values were calculated as the concentration of the compound decreasing the enzymatic activity of the untreated control by 50%. The results are in FIG. 3.

8. NRF2 Transcriptional Activity

ARE-reporter-HepG-2 cells were harvested from culture in Growth Medium 1K and they were seeded at a concentration of 40,000 cells/well into white clear-bottom 96-well microplate in 45 μL of Growth Medium 1K without Geneticin. Cells were allowed to attach overnight and then treated with different concentrations of complex 2a (from 0.01 μM to 20 μM), auranofin (from 0.01 μM to 20 μM) or DHA (from 0.01 μM to 3, 5, 10, 20 and 50 μM), and complex (from 0.01 μM to 20 μM). After incubation at 37° C. and 5% CO₂ for 16 hours, ONE-Step luciferase assay reagent (100 μL) was added and rock at room temperature for over 15 minutes. Then the luminescence of each well was determined by CLARIOstar microplate reader to quantify induction of ARE. Three independent experiments were performed as biological triplicates. The results are in FIG. 4.

9. NF-kB Transcriptional Activity

The results are in FIG. 5.

10. References
[1] R. K. Haynes, H.-W. Chan, M.-K. Cheung, W.-L. Lam, M.-K. Soo, H.-W. Tsang, A. Voerste, I. D Williams, Eur. J. Org. Chem., 2002, 1, 113-132.
[2] G. M. Sheldrick, Acta Crystallogr., 1990, A46, 467-473.
[3] G. M. Sheldrick, Acta Crystallogr., 2008, A64, 112-122.
[4] (a) H. D. Flack, Acta Crystallogr. 1983, A39, 876-881; (b) S. Parsons, H. D. Flack, T. Wagner, Acta Cryst. 2013, 869, 249-259.
[5] T. Mosmann, J. Immunol. Methods, 1983, 65(1-2), 55-63.
[6] O. Cuvillier, V. E. Nava, S. K. Murthy, L. C. Edsall, T. Levade, S. Milstien, S. Spiegel, Cell Death Differ., 2001, 2, 162-171.
[7] C Hemmert, A. Fabië, A. Fabre, F. Benoit-Vical, H. Gornitzka, Eur. J. Med. Chem., 2013, 60, 64-75.
[8] Z. Zhao, P. Gao, Y. You, T. Chen, Chem. Eur. J., 2018, 24, 3289-3298.
[9] R. Rubbiani, I. Kitanovic, H. Alborzinia, S. Can, A. Kitanovic, L. A. Onambele, M. Stefanopoulou, Y. Geldmacher, W. S. Sheldrick, G. Wolber, A. Prokop, S. Wölfl, I. Ott, J. Med. Chem., 2010, 53, 8608-8618.

1. Compound chosen from compounds of formula (I) and their isomers: 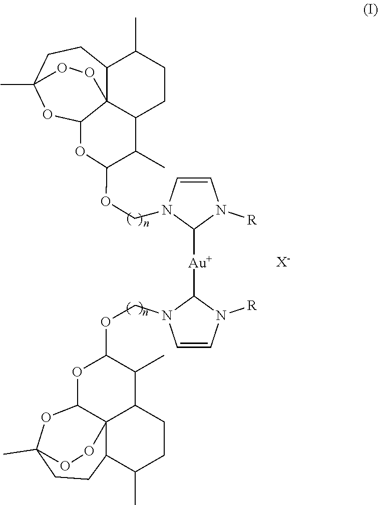
wherein each R is independently a C1-C6 alkyl, quinoline, benzyl or mesityl,
X⁻ is an anion, and
n is a integer which is equal to 3, 4 or 5.

2. Compound according to claim 1, wherein it is chosen from compounds of formula (I′): 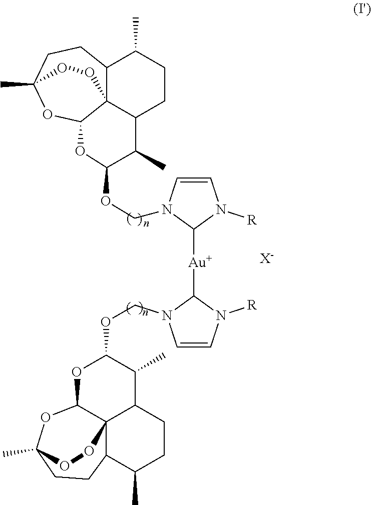
wherein each R is independently a C1-C6 alkyl, quinoline, benzyl or mesityl,
X⁻ is an anion, and
n is a integer which is equal to 3, 4 or 5.

3. Compound according to claim 1, wherein the C1-C6 alkyl is a linear hydrocarbon group comprising from 1 to 6 carbon atoms, or a branched hydrocarbon group comprising from 3 to 6 carbon atoms.

4. Compound according to claim 1, wherein the R radicals are identical or different, and are chosen from methyl, quinolone, benzyl and mesityl radicals.

5. Compound according to claim 1, wherein both R radicals are identical.

6. Compound according to claim 1, wherein X⁻ is an anion chosen from halogens, nitrate and hexafluorophosphate.

7. Compound according to claim 1, wherein the compound is chosen from the following compounds:

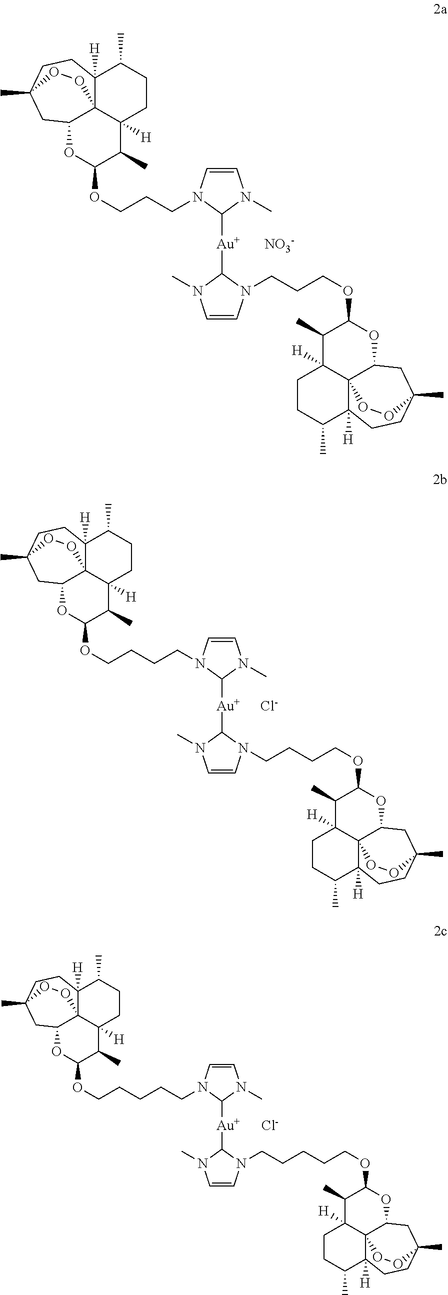

8. Composition comprising, in a pharmaceutically acceptable medium, at least one compound of formula (I) according to claim 1.

9. (canceled)

10. A method for treating and/or preventing cancer, and/or for increasing the sensitivity of a cancer to a chemotherapeutic agent, and/or for decreasing the resistance of a cancer with respect to a chemotherapeutic drug, comprising administering to a subject in need thereof with an effective amount of at least one compound of formula (I) wherein the cancer is a solid or non solid.

11. Compound of formula (IV): 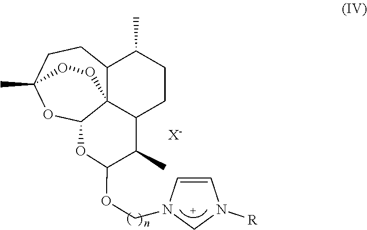
wherein:
each R is independently a C1-C6 alkyl, quinoline, benzyl or mesityl,
X⁻ is an anion, and
n is a integer which is equal to 3, 4 or 5.

12. A method for treating cancer, and/or for preventing cancer metastasis, and/or for preventing cancer recurrence, and/or for decreasing resistance to an additional therapy, in a subject, said method comprising administering to a subject in need thereof a combination product comprising:
a) a compound according to claim 1, and
b) at least one additional therapy,
wherein the compound and the at least one additional therapy are administered simultaneously, separately or sequentially.

13. The method according to claim 12, wherein said at least one additional therapy b) is immunotherapy, chemotherapy and/or radiotherapy.

14. The method according to claim 12, wherein the subject is a human suffering from a cancer and resistant to chemotherapy.

15. A method for preventing and/or treating an inflammatory disease, comprising administering to a subject in need thereof an effective amount of at least one compound according to claim 1.

16. The compound according to claim 3, wherein the C1-C6 alkyl is methyl, ethyl, n-propyl, isopropyl, n-butyl, isobutyl, tert-butyl, n-pentyl, isopentyl or n-hexyl.

17. The compound according to claim 5, wherein both R radicals are methyl.

18. The compound according to claim 6, wherein X⁻ is chloride (Cl⁻) or nitrate (NO₃⁻).

19. The method of claim 10, wherein the solid or non solid cancer is a colon cancer, a colorectal cancer, a melanoma, a bone cancer, a breast cancer, a thyroid cancer, a prostate cancer, an ovarian cancer, a lung cancer, a pancreatic cancer, a glioma, a cervical cancer, an endometrial cancer, a head and neck cancer, a liver cancer, a bladder cancer, a renal cancer, a skin cancer, a stomach cancer, a testis cancer, an urothelial cancer, an adrenocortical carcinoma, leukemia or lymphoma.
